(19) United States
(12) Patent Application Publication
(10) Pub. No.: US 2005/0244956 A1
(43) Pub. Date: Nov. 3, 2005

(54) Test culture dish

(75) Inventor: Sergey V. Brodsky, Yorktown Heights, NY (US)

Correspondence Address: NADA JAIN, P.C., 560 White Plains Road, Suite 460, Tarrytown, NY, 10591, US

(73) Assignee: New York Medical College, Valhalla, NY (US)

(21) Appl. No.: 11/120,386

(22) Filed: May 3, 2005

Related U.S. Application Data
(60) Provisional application No. 60/566,926, filed on May 3, 2004.

(51) Int. Cl. C12M 1/34

(52) U.S. Cl. 435/288.3; 435/288.4

(57) ABSTRACT

The invention relates to a novel test culture dish, a method of making thereof, and a method of uniform and simultaneous culture, maintenance, growth and/or observation of cells, cell fragments, diagnostic specimens or liquid samples or solutions generally.

This application claims the benefit, under 35 USC Section 119, of the U.S. Provisional Appl. No. 60/566,926 filed May 3, 2004, the disclosure of which is hereby incorporated herein by reference.

The invention relates to a novel test culture dish, a method of making thereof, and a method of uniform and simultaneous culture, maintenance, growth and/or observation of cells, cell fragments, diagnostic specimens or liquid samples or solutions generally.

### BACKGROUND

Existing methods for studying cell-to-cell interactions, cell surface interactions, cell adhesions and/or effects of several treatments and/or variables on cells require culturing of cells in independent chambers. Such chambers may be conveniently combined into multi-chamber plates which simplifies manipulation of several treatments. However, each chamber in such a multi-chamber plate is independent from the other chambers so that medium and/or cells must be dispensed one chamber at a time; i.e., media and/or cells cannot be dispensed uniformly and simultaneously. The drawback of such construction of multi-chamber plates is that the amount of the media and/or the number of the cells very from chamber to chamber thus adding to the experimental variability and inaccuracy. Moreover, the current construction of multi-chamber plates does not allow for simultaneous observation and comparison of several treatments under the microscope; not more than one treatment can be observed at a time.

There is a need in the art, therefore, for cell culture dishes as well as test culture dishes generally which allow for uniform distribution of media, cells, cell fragments, diagnostic specimens or liquid samples or solutions across several experimental treatments, and for simultaneous observation, study or comparison of such multiple treatments. Applicants have now discovered a test culture dish which offers these properties.

### SUMMARY OF THE INVENTION

The invention relates to a novel test culture dish, a method of making thereof, and a method of simultaneous culture, maintenance, growth and/or observation of cells or other test treatments.

In one aspect, the invention relates to a test culture dish adapted for uniform distribution of media, cells, cell fragments, diagnostic specimens or liquid samples or solutions and/or simultaneous viewing and comparison of at least two experimental treatments, which test culture dish comprises at least one chamber with at least one septum therein.

In another aspect, the invention relates to a method of making the test culture dish of the invention.

In yet another aspect of the invention, a method of uniform culturing and/or simultaneous observing and comparing of at least two experimental treatments is provided.

### BRIEF DESCRIPTION OF THE DRAWINGS

FIG. 1A-B represents a schematic view of the cell culture chamber of the invention, A represent the aerial view and B represents the side view of the chamber.

FIG. 2 represents a microscope image of HUVEC cells in the chamber of the invention divided by a septum (S) into two compartments filled with native (N) or glycated (G) collagen. Angiogenic patterns were monitored simultaneously on both sides of the septum (magnification 40×).

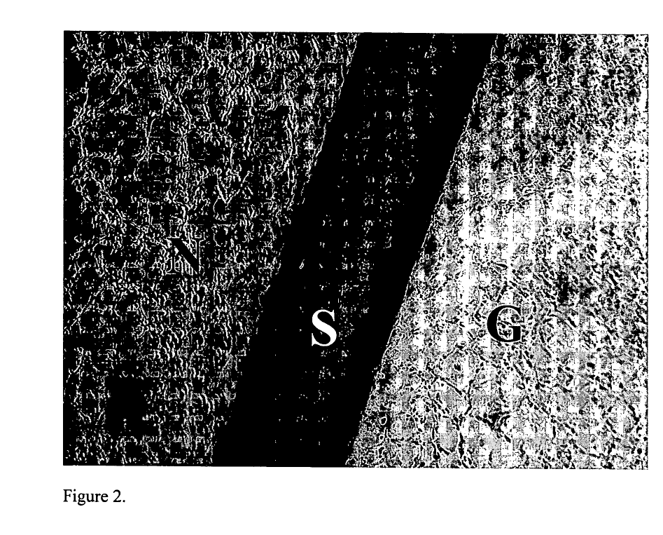

### DETAILED DESCRIPTION OF THE INVENTION

The invention relates to a novel test culture dish, a method of making thereof, and a method of uniform and simultaneous culture, maintenance, growth and/or observation of cells, cell fragments, diagnostic specimens or liquid samples or solutions. While in the preferred embodiment, the invention relates to a cell culture dish, the dish of the invention may also be useful for experimental environments which do not contain cell, for example for diagnostic purposes where cell fragments (e.g. cell membranes) or bodily fluids may be used. For example, the test culture dish of the invention may be used for immunoabsorption assays to detect the levels of substances in biological fluids or to study protein-protein interactions.

The test culture dish (e.g. cell culture dish) is adapted for uniform distribution of test material, media and/or cells and/or simultaneous viewing and comparison of at least two experimental treatments. The dish comprises at least one chamber, the chamber comprising at least one septum therein. In certain embodiments, the test culture dish comprises a plurality of chambers, each comprising one or more septums.

As used herein, a “septum” is a physical division or barrier positioned within the chamber (usually at the bottom of the chamber) providing for (i) separation of test treatments, (ii) uniform distribution of media, cells or liquid samples or solutions, and (iii) optionally unobstructed, simultaneous viewing of test results.

The height of the septum is such that it accomplishes purposes (i) and (ii) above. Thus, its minimum height is such that it sufficiently separates test treatments applied to the bottom of the chamber. Its maximum height is such that it allows for uniform distribution of media, cells, liquid samples or solutions over the entire surface of the chamber. This is accomplished by allowing the media, suspended cells, liquid samples or solutions to freely flow over the septum resulting in even distribution of such material over the entire chamber. Because the level of the media, suspended cells, liquid samples or solutions is higher than the level of the septum, two or more test variables may be simultaneously observed under a magnifying device such as a microscope.

In some embodiments, the height of the septum may be no more than the ½, or ⅓, or ¼ or ⅕ or 1/10 or 1/20 or 1/50 or 1/100 of the height of the chamber. A person of skill in the art can determine the desired septum height depending on the size and shape of the chamber keeping in mind the guidance provided herein and the purposes to be accomplish by the test culture of the invention. For example, when a shallow chamber is used, the maximum height of the septum may be less (proportionally to the height of the chamber) than when a deeper chamber is used. For example, the height of a septum suitable for a shallow chamber may be less than ½ of the chamber. As for the minimum height of the septum, a person of skill in the art can determine it such as to accomplish the separation of test treatments. Manufacturing limitations may also be taken into consideration. In some embodiments, the minimum septum height may be, for example, at least 0.1 mm, or at least 0.5 mm, or at least 0.75 mm, or at least 1 mm, or at least 2.00 mm or at least 5 mm. Any combination of the above minimum and maximum septum heights is within the scope of the invention.

In certain embodiments the width of the septum is such that it allows for the unobstructed and simultaneous observation of at least two test treatments under a magnifying device, such as a microscope. A person of skill in the art would appreciate that the higher the magnification used, the thinner the septum is preferred. For example, in certain embodiments, the septum thickness may range from 0.001 mm to 2.0 mm, or from 0.001 mm to 1 mm, or from 0.01 mm to 0.5 mm, or from 0.001 mm to 0.1 mm.

The test culture dish or vessel of the invention may be manufactured from the materials apparent to persons of skill in the art, such as for example polystyrene, polyethylene, polypropylene, polycarbonate, and polyvinyl thermoplastic resins using conventional injection-molding or thermoforming methods. Depending on the intended uses, the test culture dish may be manufactured to contain one chamber or a plurality of chambers. For example, a multi-chamber (multi-well) cell culture dish of the invention may contain between 4 and 384 chambers (wells) per dish (e.g. 4, 6, 12, 24, 48 and 96 chambers). Depending upon the size of chambers in the cell culture dish, the liquid capacity of each chamber may vary; for example the chamber may contain from about 100 microliters to about 2 milliliters. The test culture dish may also comprise a removable lid.

In a preferred embodiment, the septum is manufactured as an integral part of the test culture dish from the same material as exemplified above. However, the septum may be manufactured separately and inserted into the chamber at the time of manufacturing, assembly or at the time of use. A person of skill in the art will appreciate the various ways of securing the separately manufactured septum to the chamber. One such way of securing is described in the Example. Thus, a septum for use in a standard culture dish, for example a standard multi-chamber plate is also within the scope of the invention.

In a further embodiment of the invention, the septum described above is replaced by the pretreatment of the chamber with at least two different test reagents or the like. For example, the chamber may be pretreated by test reagents, each test reagent being applied in a separate section of the chamber.

A method of uniform culturing and/or simultaneous observing and comparing of at least two experimental test treatments is also provided. A person of skill in the art will appreciate various ways of using the test culture dishes of the invention. For example, chamber sections/divisions created by the septums may be each pretreated, treated or filled with a different test compound or any different physical, chemical or biological reagent. Cells suspended in a medium (or any other solution) may be then applied to the chamber resulting in even distribution of cells (solution) over the entire surface of the chamber. Depending on the experiment, the chamber may then be placed in an incubator device to allow for cell growth, attachment, or interaction to, with, on the test surfaces of individual chamber sections. The results may be observed and documented simultaneously under a microscope. For example, to observe cell-cell and cell-surface interactions, a magnification of 100× to 1500× may be used, for an angiogenesis assay described in the Example, a magnification of 20× to 100× may be used.

The following non-limiting Example further described the invention.

### EXAMPLE

The chamber of the invention was successfully used to study the effects of diabetic modified matrices on the angiogenic properties of Human Umbilical Vein Endothelial Cells (HUVEC).

The chambers, each containing one septum, were manufactured from a 48-well cell culture plate by inserting 1 mm height, 0.1 mm thick septums into each well (chamber). The septums were not mechanically affixed but were held in place by tension created by inserting septums longer than the diameter of the chamber (e.g. 0.5 mm longer). The septum may also be held in place by using a hard gel.

The resulting two compartments within each chamber (well) were filled with 100 μl of either native or glycated collagen I, and allowed to solidify for 30 min at 37° C. About 150,000 HUVEC in 0.5 ml of EBM-2 medium supplemented with 2% FBS were seeded into the chambers. The cell were then incubated at 37° C. in 95% air plus 5% CO₂ for four hours and then observed under a microscope.

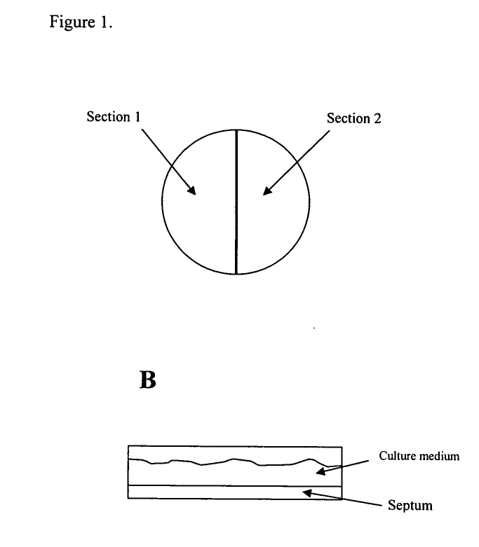

The progress of angiogenesis on both test treatments was monitored simultaneously by observing both sides of the septum using a Nikon TE2000-U microscope (40× magnification) equipped with a CCD camera (Diagnostic Instruments, Sterling Heights, Mich.) (FIG. 1).

Referring to FIG. 1, S is a chamber septum, N is a test treatment with native collagen, and G is a test treatment with glycated collagen. Due to the same level of the medium on both sides of the septum and the small width of the septum, two experimental treatments could be simultaneously observed and documented.

1. A test culture dish adapted for uniform distribution of test material over experimental variables and/or simultaneous observation of experimental progress and/or results comprising at least one chamber, which chamber comprises at least one septum.

2. The test culture dish of claim 1, which is a cell culture dish.

3. The test culture dish of claim 2 comprising a plurality of chambers.

4. The test culture dish of claim 2, wherein the chamber comprises a plurality of septums.

5. The test culture dish of claim 1, wherein the septum is no more than 10 mm high.

6. The test culture dish of claim 2, wherein the septum is no more than 10 mm high.

7. The test culture dish of claim 1, wherein the thickness of the septum is in the range of from 0.001 mm to 2 mm.

8. The test culture dish of claim 2, wherein the thickness of the septum is in the range of from 0.001 mm to 2 mm.

9. A test culture dish adapted for uniform distribution of test material over experimental variables and/or simultaneous observation of experimental progress and/or results comprising at least one chamber, which chamber comprises at least two differently pretreated sections.

10. The test culture dish of claim 9, wherein the pretreated sections are divided by a visual division.

11. A septum for use in a test culture dish, wherein the septum is adapted to provide uniform distribution of test material over experimental variables and/or simultaneous observation of experimental progress and/or results when inserted in a chamber of a test culture dish.

12. The septum of claim 11, wherein the test culture dish is a cell culture dish.

13. The septum of claim 12 having the height in the range of 0.1 mm to 5 mm.

14. The septum of claim 12, having the width in the range of 0.001 mm to 2 mm.
